(19) United States
(12) Patent Application Publication
(10) Pub. No.: US 2006/0080150 A1
(43) Pub. Date: Apr. 13, 2006

(54) Whole-patient system for joint replacement care

(75) Inventors: Robert L. Patterson III, Medford, OR (US); Kai R. McSwain, Medford, OR (US)

Correspondence Address: THE WEBB LAW FIRM, P.C., 700 KOPPERS BUILDING, 436 SEVENTH AVENUE, PITTSBURGH, PA, 15219, US

(73) Assignee: Southern Oregon Orthopedics, Inc., Medford, OR (US)

(21) Appl. No.: 11/240,653

(22) Filed: Sep. 30, 2005

Related U.S. Application Data
(60) Provisional application No. 60/614,499, filed on Sep. 30, 2004.

(51) Int. Cl. A61B 5/00

(52) U.S. Cl. 705/3

(57) ABSTRACT

A whole-patient system for joint replacement care has seven intrinsic steps/features, which apply both to the patient care method itself and to the overall administrative system which embodies the method. These key seven steps are: (1) Patient evaluation and pre-screening; (2) Body Mass Index assessment; (3) Diagnostic study selection protocol; (4) Operating room set-up protocol; (5) Joint size and type selection and delivery; (6) Procedure length duration estimation; and (7) Post-operative nausea/vomiting and pain management. These steps are institutionalized into a business method in which each is particularly documented.

### CROSS-REFERENCE TO RELATED APPLICATION

This application claims the benefit of, and incorporates herein by reference. U.S. Provisional Application No. 60/614,499, filed Sep. 30, 2004 entitled, “Health Care Products.”

### BACKGROUND OF THE INVENTION

1. Field of the Invention

The invention pertains to a start-to-finish system to improve the overall outcomes and patient and doctor satisfaction when joint replacement procedures are indicated and performed.

2. Description of Related Art

Joint replacement surgery has not traditionally been thought to be a mature discipline of medicine, even in recent years. There are several reasons for this. Materials appropriate to the requirements for prosthetic implants have only very lately approached the wear-resistance and biochemical neutrality required for long term patient satisfaction. Fine appreciation for biomechanics has only recently changed the way surgeons believe prosthetics should be designed and implanted. Major surgery has been and still is fraught with grave risks including mortality, which risks understandably have discouraged many patients from having joint replacement procedures, or have encouraged at least the postponement of those procedures. Postponing an indicated procedure unfortunately increases mortality and other risks in many cases. Joint replacement requires selection of prosthetic joints of the correct size, which creates inventory and delivery problems and, on occasion, mistakes occur in the selection of prosthetics of the wrong or at least non-optimal dimensions. Patient rehabilitation from any major surgery is difficult enough, but joint replacement poses the greatest rehabilitation challenge in terms of pain management issues and the extent and nature of the required physical therapy. Objectively considering the hurdles posed by joint replacement procedures, a layman readily admits to surprise upon learning that so many joint replacement procedures are ultimately successful despite the multiple risks.

Even after joint replacement prostheses and protocols achieved satisfactory quality, many challenges to efficient, economic whole-patient care have remained, however. The development of high-quality prostheses with long wear-resistance means that a range of prosthesis grades is now available. For example, at this writing there are standard knee prostheses available in at least three grades having retail price tags of about $4300 for the most basic but fully acceptable version, about $5100 for an enhanced model, and about $6700 for the longest-wearing, best grade available. Surgeons themselves often silently determine the best choice for a given patient, and those choices can be obvious and intuitive: a young man or woman who still actively plays competitive sports needs the long life of the best possible prosthesis whereas the basic, fully acceptable model may be completely adequately for an older individual and/or an individual whose chosen lifestyle is predominantly sedentary. Neither a surgeon nor a hospital administrator would want such a choice to be made on either basis of availability or profitability, for ethical and efficiency reasons, and for insurance reimbursement purposes it is best to have the selection protocol and surgeon's decision in writing. Even apart from choice of grade, choice of prosthesis size is an enormous challenge in selecting and implanting a replacement joint: a prosthetic joint which is too large or too small will never be satisfactory no matter how determined are the post-operative pain management and physical therapy initiatives. Patient evaluation prior even to scheduling the procedure has become more challenging with time, too. Increasingly older patients are treated with joint replacement at the present time, just as thoracic surgery is now commonly performed on individuals in their 80s and even 90s. Appropriate screening of patients prior to joint replacement is thus more important than ever. Cardiac or pulmonary risks must be assessed and, if necessary, the patient referred for appropriate evaluation by a cardiologist or pulmonologist before any other procedures are performed. Perhaps most lamentably of all, it has not heretofore even been considered to address all the various above-mentioned problems all together, because each of them has been treated individually, or reactively, without there ever having been a stated goal to find a comprehensive system to address all these problems in a comprehensive way.

For the above and other reasons, a need persists for an overall system in which every ramification of joint replacement procedure is coordinated for a given patient. Such a system maximizes quality, efficiency, economy, care of the whole patient including pain management, postoperative nausea abatement, and physical therapy, in a way which maximizes patient outcome, minimizes the length of hospital stay and reduces or eliminates opportunities for waste or error.

### SUMMARY OF THE INVENTION

In order to meet this need, the present whole-patient system for joint replacement care has seven intrinsic steps/features, which apply both to the patient care method itself and to the overall administrative system which embodies the method. These key seven steps are: (1) Patient evaluation and pre-screening; (2) Body Mass Index assessment; (3) Diagnostic study selection protocol; (4) Operating room set-up protocol; (5) Joint size and type selection and delivery; (6) Procedure length duration estimation; and (7) Post-operative nausea/vomiting and pain management. There are no known instances in the prior art in which such start-to-finish patient care has been attempted or even conceived, nor any way in which the economies and quality control of such a system have been coordinated. While additional features to the overall system can be added, the above seven intrinsic steps/features form the heart of the present invention.

### BRIEF DESCRIPTION OF THE DRAWING(S)

FIG. 1 is the first page of a four page form which identifies basic patient joint replacement information including Body Mass Index and the name and facsimile number of the patient's Primary Care Provider, among other information;

FIG. 2 is the second page of a four page form typical for use in the fourth and fifth step/features of the invention, namely, operating room set-up and joint size and type selection and delivery;

FIG. 3 is the third page of a four page form typical for use in the third step/feature of the present invention, namely, the Diagnostic study selection protocol; and

FIG. 4 is the fourth page of a four page form typical for use in initial patient evaluation and screening, which provides a sample “Preoperative Cardiopulmonary Risk Assessment (POCA) Patient Questionnaire.”

### DESCRIPTION OF THE PREFERRED EMBODIMENT(S)

The easiest way to comprehend the present invention is to consider each of its seven steps/features individually, with the understanding that a medical/surgical management system and method results for optimized patient care before, during and after joint replacement procedures. This invention and its proprietary nature is not in any way intended to constrain what an individual physician or surgeon may individually do: the proprietary nature of this invention is in the administrative method and the hybrid physician/hospital administration system which is in itself a business method type invention intended for institutional implementation.

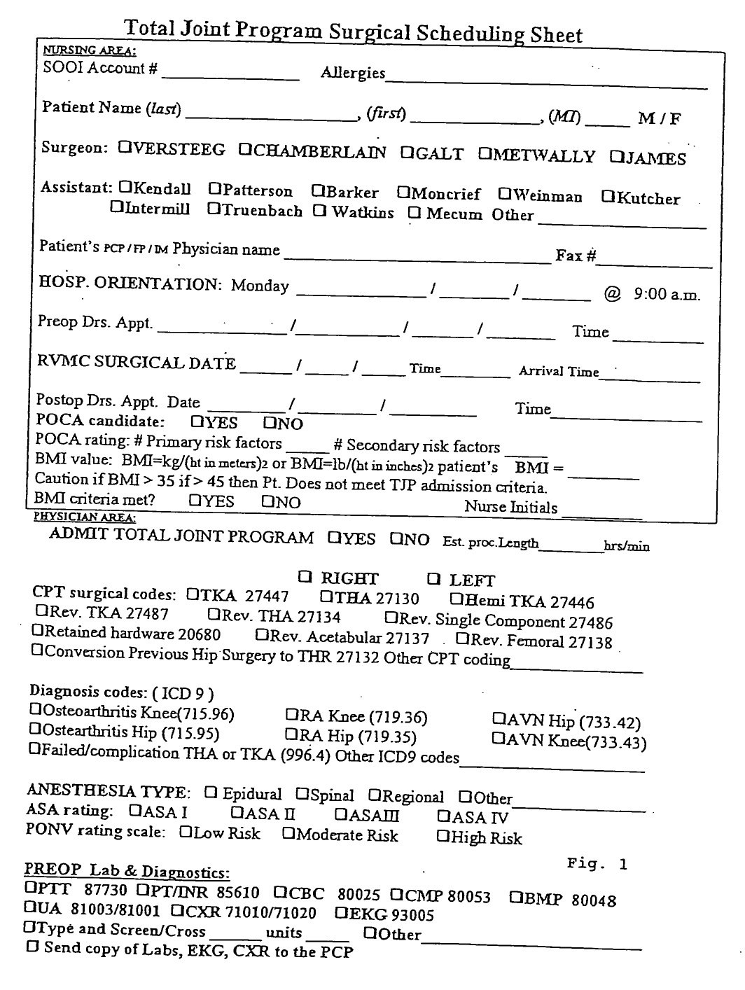

Patient evaluation and screening constitute the first of the key seven features of the present method and system. Traditionally, prior to joint replacement, the surgeon would refer the patient to a primary care provider (PCP) for a thorough pre-operative examination. However, for certain patients with ongoing care from their personal PCP, it is often possible for the PCP to provide the necessary medical clearance with an abbreviated physical examination or, in some cases, with no separate physical examination at all. In other words, there are many patients whose health care is so consistent and ongoing that the patient's PCP can vouch for the patient's being strong enough and healthy enough for successful joint replacement based on contemporaneous information available to the physician. Certain patients are obviously healthy enough for joint replacement procedures that the surgeon who will perform the joint replacement can assess the health status of the candidate without consulting the PCP at all. Therefore, referring now to FIG. 1, the provision of the PCP's name and facsimile number, in a prominent position on the form exemplary for use in initial patient evaluation and screening, facilitates sending a facsimile request for medical clearance to the PCP only if and when the solicitation of such PCP medical clearance is necessary.

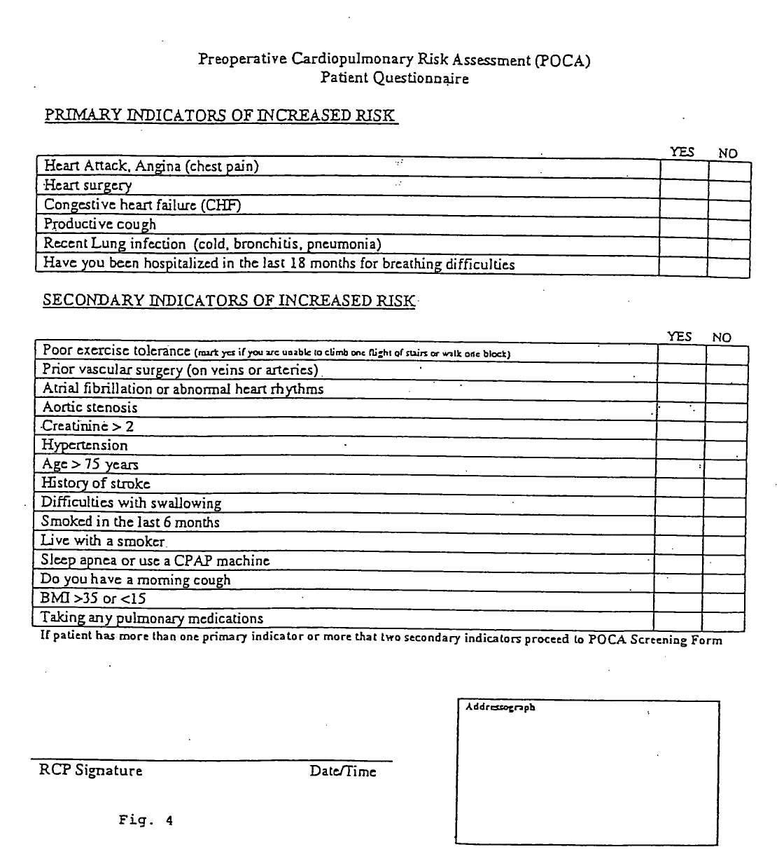

Having said that, however, Preoperative Cardiopulmonary Risk Assessment (POCA) is a test that is implemented before the PCP is solicited with a standard medical clearance form. The POCA Patient Questionnaire may approximate a form typified by FIG. 4. POCA questioning itself is not proprietary to the present inventors, but the present system institutionalizes and makes comprehensive the administration of POCA with an appropriate and convenient form and an overall system which affirmatively requires POCA consideration. POCA recognizes typical risk scoring in which, say, a patient with more than one primary risk indicator and more than 2 secondary risk indicators is designated a POCA candidate, which in turn initiates POCA screening. POCA screening can involve electrocardiogram, chest X-ray, the Forced Expiratory Volume over 1 Second (FEV1) test and possibly other tests, and POCA status for the patient is evaluated as pass/fail. Patients who fail the POCA screening are referred to a cardiologist or pulmonologist (or other specialist) before further investigation of the feasibility of joint replacement is considered. Patients who pass the POCA screening are made the subject of a medical clearance request from each individual PCP if the surgeon deems a PCP medical clearance request to be necessary (see below).

Notwithstanding POCA screening and medical clearance by PCPs, it should not be overlooked that surgeons are physicians, too, and fully capable of evaluating a patient's candidacy for surgery. In conjunction with the above, then, the present invention also provides for “ASA” implementation, based on the criteria of the American Society of Anesthesiologists' scale of patient evaluation. An “ASA1” patient is a “healthy” patient whom the surgeon clears directly for the joint replacement procedure. An “ASAII” patient is a “moderately healthy” patient for whom the surgeon uses his or her own discretion whether the medical clearance from the PCP is necessary. An “ASAIII” patient is “a patient with health risks” whom, because the surgeon has determined, based on past medical history and current medical problems, that the patient possesses multiple co-morbid factors and who thus requires a minimum of PCP evaluation. An “ASAIV” patient is an “unhealthy patient with significant risks” whom the surgeon has deemed a significant risk of compromise, and whom requires a minimum of a specialist consultation and possible confirmatory PCP evaluation. There is deliberate built-in redundancy in these patient evaluation and screening procedures, because POCA screening by the surgeon, ASA screening by the surgeon, possible examination by a specialist and/or medical clearance evaluation by the patient's PCP maximize the likelihood of wise candidate evaluation. This step of the invention recognizes the Achilles Heel of joint replacement: there is no success if the mechanics of the joint replacement procedure seem to go well but the patient then does not recover appropriately due to pre-existing reasons that were under- or unappreciated. The thorough pre-operative assessment of patient status and risk factors cannot be overemphasized as a key to joint replacement program success. (Knowing this, versus implementing a program to assure it, are entirely different things.)

Along the same lines, while post-operative nausea/vomiting and pain management constitutes the seventh step/feature of the present invention, initial patient screening and evaluation necessarily assess post-operative nausea and vomiting long before the joint replacement procedure begins. This advance planning step is typical of all the other coordinated steps of the present inventive system. At the time of pre-operative evaluation, a pre-operative checklist for Post-Operative Nausea and Vomiting (PONV) risk factors is used to evaluate the patient. This evaluation may be conducted with or without a form to make it easier, but it must be conducted. The checklist includes patient factors which tend to suggest that nausea will be a problem. These patient factors can include, by way of example, but not limited to, that the patient is female; a non-smoker; has motion-sickness history; has post-operative nausea and vomiting history; has age <50; has history of anxiety; has Body Mass Index (BMI) >30; or has hemodynamic instability (heart rate <60 and/or systolic blood pressure <100). In addition to the patient factors, the surgical factors which tend to predict PONV are planned intra- or post-operative narcotics; shoulder or knee surgery; use of general anesthesia; and duration of procedure >1 hour. Of all the above factors, low risk is indicated by fewer than four of the above factors; 4-7 indicates moderate risk and warrants administration of Dexamethasone 8 mg pre-operatively and 4 mg Ondansetron intra-operatively; and 8-12 factors means high risk and recommended administration of 8 mg Dexamethasone pre-operatively and both 4 mg Ondansetron intra-operatively and 10 mg Metoclopramide intra-operatively. The present comprehensive system thus provides for amelioration of post-operative nausea and vomiting right from the point of initial patient evaluation, rather than waiting for post-operative nausea and vomiting to occur. The benefits of this foresight-based approach to nausea and vomiting control are enormous: patient satisfaction, pain management, healing and rehabilitation are all expedited tremendously if nausea and vomiting need not be addressed after-the-fact.

The second step/feature of the invention is the evaluation of the Body Mass Index (BMI) of the patient. FIG. 1 identifies, near the center of the exemplary form it depicts, thumbnail summaries of BMI criteria and calculation and assessment of the patient on the basis of BMI. BMI assessment prevents overweight and care-intensive patients from being assigned, absent extenuated circumstances, to a short-term (i.e., five day) program which would probably not be a good fit for their needs. Higher BMI joint replacement candidates typically require more support and physical therapy post-operatively than lower BMI candidates do. Joint replacement programs of the prior art did not facilitate screening for this important factor.

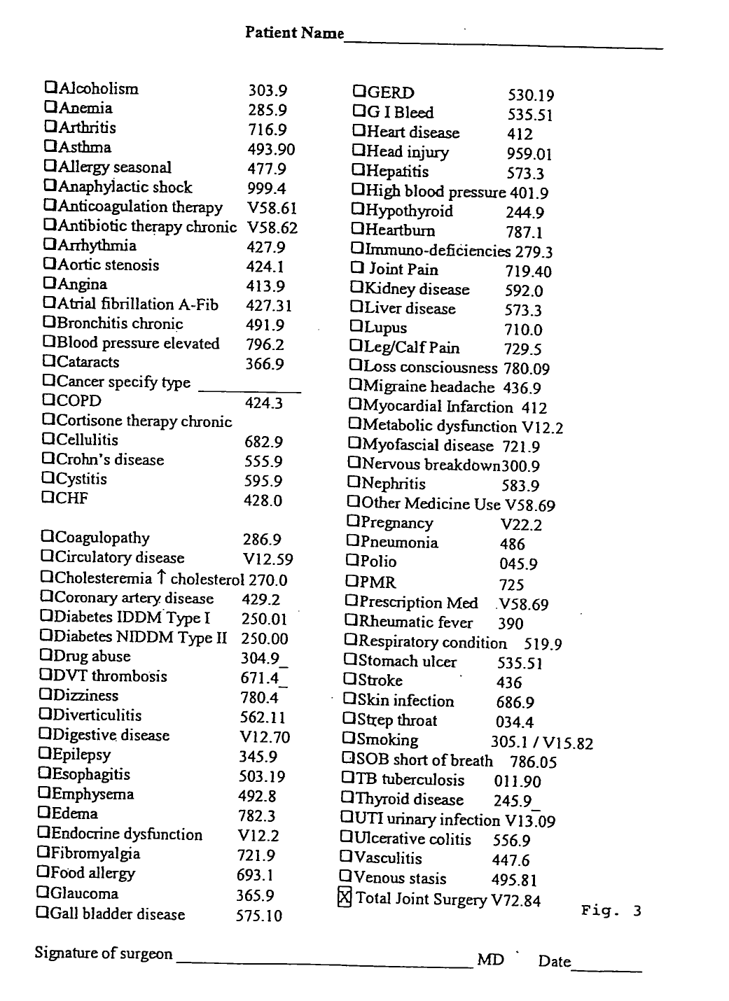

The third step/feature of the present invention is the Diagnostic study selection protocol, i.e., the selection and documentation of pre-operative diagnostic tests. FIG. 3 represents a typical diagnostic study form which lists virtually all the diagnostic tests, and their corresponding codes, that surgeons can order in the context of evaluating a patient for joint replacement. This form alone represents an extensive implementation of trial and error innovation to document the erstwhile mental menu of diagnostic tests considered by surgeons for evaluation of their potential patients. Those familiar with hospital administration recognize immediately that the corresponding code for a diagnostic test is essential, because software matches code to code and, without a code, the corresponding invoice cannot be finalized. In other words, insurance companies understandably insist on justification for a diagnostic test prior to authorizing reimbursement, so the use of a checklist such as FIG. 3 provides a consolidated document, signed by the physician, that commemorates whom ordered each test and when. The fact that the diagnostic test(s) are ordered incident to the inventive Diagnostic study selection protocol provides the justification for the test(s) inherently: the diagnostic test(s) were ordered preparatory to joint replacement surgery, during the Diagnostic study selection protocol step/feature of the overall joint replacement system.

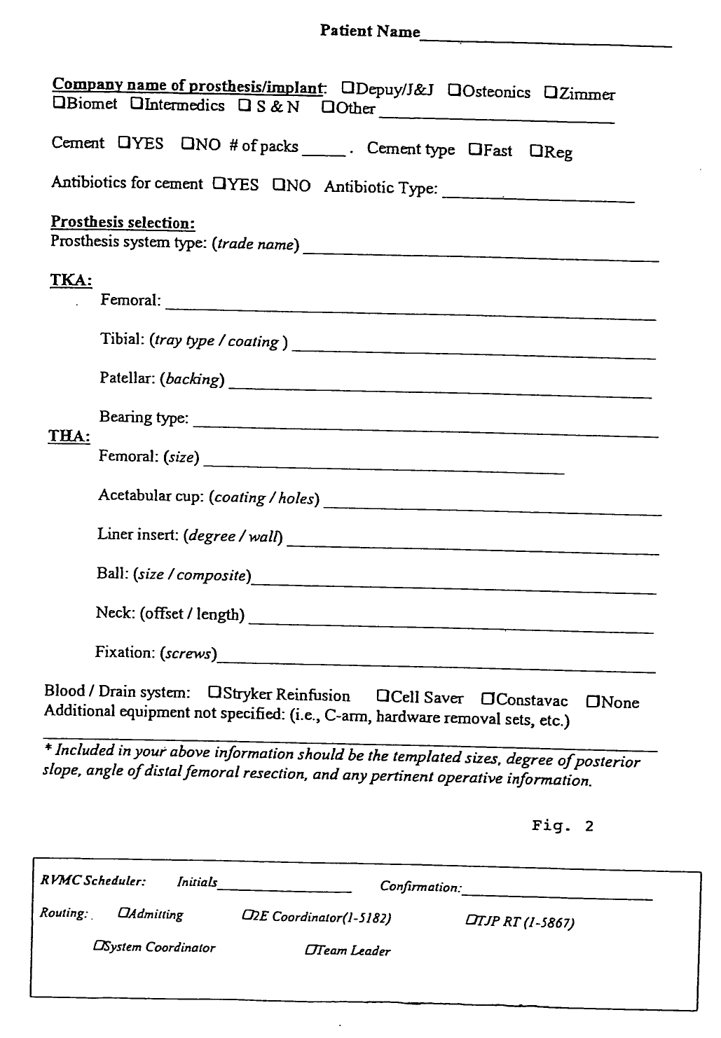

The fourth step/feature of the present invention is the operating room set-up protocol. In large part, the operating room set-up protocol is guided by use of a form (such as that shown in FIG. 2) whereby the materials and equipment that are ordered will be in the room, and those that are not ordered or needed will not be present. For example, a form such as FIG. 2 obtains documented instruction of, without giving exhaustive examples, the company name of the prosthesis/implant; and whether to provide bone cement, and if so how much and of what type. FIG. 2 will be used largely in conjunction with consideration of the joint size and type selection and delivery feature of the invention, discussed immediately below. Written instructions to outfit the operating room in right-hand or left-hand orientation are helpful, too.

The fifth step/feature of the present invention is the joint size and type selection and delivery feature of the invention. While the following protocol can be described using a standardized form, a form is not necessary as long as a writing is created, and joint size and type selection and delivery takes the following into consideration. On a quarterly basis, the surgical subcommittee of the Total Joint Program of the invention meets to review prosthesis implantation patterns. During this review each surgeon will analyze their prosthesis usage and reflect upon the appropriateness of selection. The purpose of this review is by no means intended to serve as a tool for compliance. While it is the goal of the subcommittee to make implant selection more patient-specific based on the demands of the patient, surgeon discretion remains. The guidelines of the subcommittee bear in mind two parallel categories: age categories and quality of life categories. The age categories (although this categorization is rebuttable—see below) are “under 65 years of age;” “between 65 and 80 years of age,” and “greater than 80 years of age.”

Generally speaking, the above three categories correlate with, respectively, 1) current employment, physically active with advanced recreational activity, high demand and capacity, healthy adult with low or no morbidity, and uncompromised daily activity level; 2) semi-retired, moderate recreational activity, average physical demand and capacity, moderate health and morbidity, and advanced activities of daily living; and 3) retired, little or no recreation, hobbies or physically demanding activity, low physical demand and capacity, significant morbidity and poor health with obvious length of life limitations, and ability to accomplish basic activities of living. Because these general categories do not apply to everyone, however, it is important according to the present invention to go beyond the above and to score individual patients with a quality of life evaluation scale in five categories A-E. Under category A, the patient receives 3 points for current employment, 2 points for semi-retired employment status and 1 point for retired status. Under category B, the patient receives 3 points for physically active status with advanced recreational activity, 2 points for moderate physical activity, and 1 point for little or no recreational activity. In category C, the patient receives 3 points for high physical demand and capacity, 2 points for average physical demand and capacity, and 1 point for low physical demand and capacity. In category D, the patient receives 3 points for healthy status with low or no morbidity, 2 points for moderate health and morbidity, and 1 point for significant morbidity, poor health or length of life limitations. Finally, in category E, the patient receives 3 points for uncompromised daily activity level, 2 points for advanced activities of daily living, and 1 point for accomplishing the basic activities of daily living.

Completion of the above-described quality of life evaluation scale provides the surgeon with a numerical quality of life cumulative value that is crucially patient specific. This calculated value assists the surgeon in selecting the most appropriate prosthesis for each patient. By adding the points from the above five categories, a more appropriate assignment to category 1, 2 or 3 can be accomplished rather than assigning one of three categories based on age alone, because not all age-equivalent patients are alike by any means. A patient with more than 10 points would be considered to be in Category 1, therefore, even if the patient is greater than 65 years old. A patient with 6-10 points would be assigned to Category 2, even if the patient is 65 years old or less. A patient with less than or equal to 5 points would be assigned to Category 3, again regardless of age alone. These three categories thus tend to indicate the best selection of the top, medium and functional grades of prostheses available for hip or knee replacement, respectively, with a patient specific evaluation system.

The sixth step/feature of the invention embodies the prediction of the length of the procedure. After selection of the joint manufacturer type and size, and evaluation of the patient, the surgeon based on his or her experience can project the approximate time the procedure will take. This is important information, and is annotated on a form such as that of FIG. 1, for two reasons: the anesthesiologist benefits by knowing how much anesthesia will likely be needed for how long; and the hospital administrators and operating room personnel can outfit and schedule the operating rooms accordingly, for maximum use of operating room assets.

Finally, the seventh key step/feature of the present invention is the management of post-operative nausea, vomiting and pain. Without going into great background detail here, the management of nausea and vomiting, and pain, can prove to be the key factors in a patient's successful recovery and rehabilitation. The patient who is nauseated or vomiting or in pain will not be able to participate fully (or at all) in: resumption of oral nutrition and hydration and ambulatory elimination; resumption of activities of daily living including bathing and grooming; physical therapy (which can and must be intensive for a joint replacement patient); emotional reconnection with family members and/or enjoyment and satisfaction upon receiving visitors; or resumption of normal sleep cycles, to name just a few. Poor performance of any of these post-operative activities compromises healing and wellness, yet nausea, vomiting and pain will obstruct all of them and more, with disastrous results in post-operative progress. Also, neurologists report that unremitting pain creates a kind of pain memory, so that a patient who has experienced severe pain will continue to have severe pain even after the source of the pain has been removed or resolved, and such memory-type pain can be intractable to alleviate. If pain management requires severe measures such as higher-than normal doses of narcotics or analgesics, physical therapy and other patient activities will likewise be compromised. The criticality of finessed management of post-operative control nausea, vomiting and pain simply cannot be overstated.

In order to implement the seventh step/feature of nausea/vomiting and pain management, therefore, two things happen for each patient. First, as described above, the patient receives pre-operative evaluations (PONV) and is treated pre-operatively and/or intra-operatively, if warranted, to ameliorate possible nausea even before it starts. As in so many other aspects of the present system (and all of medicine), an ounce of prevention is worth a pound of cure. Second, the program requires alert observation for post-operative nausea or vomiting despite the pre-operative risk screening, and immediately intervention to relieve the nausea or vomiting if it occurs despite all precautions. Finally, the implementation of patient-controlled analgesia (self-administration of analgesia as moderated by computer) is intrinsic to the present system and all patients are advised as to the proper use of patient controlled analgesia during pre-operative education. When pain and nausea are reduced or eliminated, normal functioning and physical therapy proceed at maximum pace, and hospital stays are minimized and patient outcomes and satisfaction are maximized. These important results cannot be left to chance and arbitrary physician and hospital staff memory: they must be achieved by a documented and institutionalized system according to the present invention.

There are other aspects of whole-patient care which can be implemented in associate with this or any other start-to-finish patient care protocol, but the above seven features are integral to the practice of the present system and method invention. Notwithstanding any of the above, however, the invention is only to be considered to be limited insofar as is set forth in the accompanying claims.

The invention claimed is:

1. A hybrid physician/hospital administration system for documented management of the joint-replacement candidate, comprising at least the seven features of: patient evaluation and pre-screening according to criteria including at least preoperative cardiopulmonary assessment and ASA categorization; Body Mass Index assessment; Diagnostic study selection and documentation; operating room set-up by specifying at least a kind and type of prosthesis and an amount and type of cement on an appropriate written order; implementation of a scoring protocol for joint size and type selection and delivery and written orders for a particular prosthesis size prior to the procedure; projection and documentation of projected length of operating room procedure; and pre-operative screening and post-operative management of nausea, vomiting and pain.

2. The hybrid physician/hospital administration system according to claim 1 wherein said ASA categorization includes possible patient assignment to one of the categories ASAI, ASAII, ASAIII or ASAIV.

3. The hybrid physician/hospital administration system according to claim 1 wherein the scoring protocol for joint size and type assesses point values for each of employment status, customary physical activity status, customary physical demand status, health status, and activity level.

4. The hybrid physician/hospital administration system according to claim 1, wherein the pre-operative screening for nausea and vomiting considers and, where indicated, implements the pre-operative and intra-operative administration of one or more anti-nausea pharmaceuticals even before any nausea has had a chance to occur.

5. The hybrid physician/hospital administration system according to claim 1, wherein the pre-operative screening for Body Mass Index (BMI) assesses whether the BMI is greater than 35.

6. The hybrid physician/hospital administration system according to claim 1, wherein the pre-operative screening for Body Mass Index (BMI) assess whether the BMI is greater than 45 and, if so, the patient is identified as an extremely high risk candidate for joint replacement.

7. A method for managing a joint replacement candidate, comprising the steps of: evaluating the patient pre-operatively including at least assessment of preoperative cardiopulmonary criteria and ASA categorization; evaluating the Body Mass Index of the patient; identifying and documenting on a single signed form the diagnostic studies needed for the patient prior to the joint replacement procedure; implementation of a check-list driven protocol for setting up the operating room to include a particular make and model of joint and what type and how much of bone cement; selecting a joint type and size based on a scoring system to determine patient need; projecting and documenting the anticipated length of the procedure; and pre-operative screening and post-operative management of nausea, vomiting and pain.
